(19) United States
(12) Patent Application Publication
(10) Pub. No.: US 2024/0016381 A1
(43) Pub. Date: Jan. 18, 2024

(54) DEVICE AND METHOD FOR NON-CONTACT MEASUREMENT OF AN INTRAOCULAR PRESSURE

(71) Applicant: N.M.B. MEDICAL APPLICATIONS LTD, Herzliya (IL)

(72) Inventors: Oren GLOBERMAN, Kfar Shmaryahu (IL); Mordechay BEYAR, Tel Aviv (IL); Danielle VALES, Tel Aviv (IL)

(73) Assignee: N.M.B. MEDICAL APPLICATIONS LTD, Herzliya (IL)

(21) Appl. No.: 18/253,055

(22) PCT Filed: Nov. 14, 2021
(86) PCT No.: PCT/IL2021/051351

§ 371 Date: May 16, 2023

Related U.S. Application Data
(60) Provisional application No. 63/114,560, filed on Nov. 17, 2020.

(51) Int. Cl. A61B 3/16

(52) U.S. Cl. CPC A61B 3/165

(57) ABSTRACT

A device for a non-contact measurement of an IOP of a subjects eye including: a frame; a conduit connected to the frame and aligned along a conduit axis; at least one camera connected to the frame and configured to capture one or more image frames each comprising an image of a side view of a cornea of the subjects eye; a fluid pulse generator in fluid communication with the conduit, the fluid pulse generator configured to generate a fluid pulse and puff the fluid pulse through the conduit; and a controller comprising a processing unit, the processing unit configured to: receive the one or more image frames; determine one or more fluid pulse pressure values; and determine the IOP value of the subjects eye based on at least one of the one or more image frames and at least one of the one or more fluid pressure values.

### FIELD OF THE INVENTION

The present invention relates to the field of ophthalmology and, more particularly, to non-invasive and non-contact devices and methods of measurement of intraocular pressure.

### BACKGROUND OF THE INVENTION

Current techniques of measuring the intraocular pressure (IOP) of a subject typically require direct contact with an eye of the subject. The contact may be achieved either by pressing against a cornea of the eye with an applicator (e.g., such as in Goldmann Applanation Tonometer) or by pressing against the cornea with an air jet to create local physical depression of the cornea that is further translated into an IOP value. However, such techniques typically require technical skills and cannot be performed by the subject him/herself.

### SUMMARY OF THE INVENTION

Some embodiments of the present invention provide a device for a non-contact measurement of the intraocular pressure (IOP) of a subject's eye, the device including: a frame; a conduit connected to the frame and aligned along a conduit axis; at least one camera connected to the frame and configured to capture one or more image frames, each image frame including an image of a side view of a cornea of the subject's eye; a fluid pulse generator in fluid communication with the conduit, the fluid pulse generator being configured to generate a fluid pulse and to puff the fluid pulse through the conduit; and a controller including a processing unit, the processing unit being configured to: receive the one or more image frames; determine one or more fluid pulse pressure values; and determine the IOP value of the subject's eye based on at least one of the one or more image frames and at least one of the one or more fluid pressure values.

In some embodiments, the processing unit is configured to: detect, based on at least one of one or more image frames, that the cornea of the subject's eye is in its first applanated state; and determine a fluid pulse pressure value at which the cornea has reached its first applanated state, wherein the determined fluid pulse pressure value is indicative of the IOP value of the subject's eye.

In some embodiments, the processing unit is configured to: determine a fluid pulse pressure value that would have caused the cornea of the subject's eye to reach its first applanated state based on a known deformation-pressure variation pattern of the subject's eye, based on a subset of the one or more image frames and based on at least one of the one or more fluid pulse pressure values, wherein the determined fluid pulse pressure value is indicative of the IOP value of the subject's eye.

In some embodiments, the camera is connected to the frame such that a field-of-view of the camera is located along an axis that is perpendicular to the conduit axis and is substantially parallel to a tangent to the cornea when the conduit is aligned with the cornea along the conduit axis.

In some embodiments, the processing unit is configured to determine the one or more fluid pulse pressure values based on readings of a pressure sensor configured to measure a fluid pulse pressure within the conduit.

In some embodiments, the processing unit is configured to determine the one or more fluid pulse pressure values based on a known pressure-time variation pattern of the fluid pulse and a time point at which the fluid pulse generator has puffed the fluid pulse through the conduit.

In some embodiments, the device may include: a first camera configured to capture one or more image frames of the side view of the cornea; and a second camera configured to capture, at a predefined time shift with respect to the first camera, one or more image frames of the side view of the cornea; wherein the processing unit is configured to combine the one or more image frames captured by the first camera with the one or more image frames captured by the second camera into a single image dataset.

In some embodiments, the device may include a reference marker connected to the frame such that the one or more image frames being captured by the camera include an image of at least a portion of the reference marker, the reference marker includes at least one of: a visual length marker, a visual position marker and a visual color marker.

In some embodiments, the device may include a conduit positioning mechanism configured to at least one of: controllably move the conduit with respect to the frame along the conduit axis; controllably move the conduit with respect to the frame in one or more directions that are perpendicular to the conduit axis; and controllably rotate the conduit with respect to the frame about at least one axis that is perpendicular to the conduit axis.

In some embodiments, the one or more image frames further include an image of an outlet conduit end, wherein the processing unit is configured to determine a distance between the outlet conduit end and the cornea based on the one or more image frames.

In some embodiments, the controller is configured to: control the fluid pulse generator to puff the fluid pulse if the outlet conduit end is at a predefined distance from the cornea; and prevent from the fluid pulse generator to puff the fluid pulse if the outlet conduit end is not at a predefined distance from the cornea.

In some embodiments, the controller is configured to control the conduit positioning mechanism so as to position the outlet conduit end at a predefined distance from the cornea.

In some embodiments, the device may include an additional camera connected to the frame and configured to capture one or more additional image frames, each including an image of a front view of the cornea of the subject's eye.

In some embodiments, the processing unit is configured to verify that the conduit is radial to a center of the cornea based on the one or more additional image frames and a known position of the conduit with respect to the frame.

In some embodiments, the controller is configured to: control the fluid pulse generator to puff the fluid pulse upon the verification that the conduit is radial to the center of the cornea; and prevent from the fluid pulse generator to puff the fluid pulse if the conduit is not aligned with the center of the cornea.

In some embodiments, the controller is configured to control the conduit positioning mechanism so as to align the conduit with the center of the cornea.

In some embodiments, the processing unit is configured to detect a blinking of the subject's eye based on the one or more image frames, wherein the controller is configured to control the fluid pulse generator to puff the fluid pulse through the conduit at a predetermined time interval after the blinking of the subject's eye ends.

In some embodiments, the device may include a light source configured to emit a light beam alignable along the conduit axis so as the subject can align the subject's eye with the conduit axis.

In some embodiments, the device may include light condition control means so as to cause the subject's eye to be exposed to substantially the same lighting conditions each time the device is used.

In some embodiments, the device may include one or more movement sensor sensors connected to the frame, wherein the processing unit is configured, based on readings of the one or more movement sensors, to at least one of: determine an orientation of the device in a reference coordinate system; determine whether a subject's right eye or a subject's left eye is being tested by the device; determine whether or not the device is stationary; and determine that the fluid pulse generator has puffed the fluid pulse.

In some embodiments, the controller is configured to: control the fluid pulse generator to puff the fluid pulse upon verification that the device is horizontal; and prevent from the fluid pulse generator to puff the fluid pulse if the device is not horizontal.

In some embodiments, the controller is configured to: control the fluid pulse generator to puff the fluid pulse upon verification that the device is stationary; and prevent from the fluid pulse generator to puff the fluid pulse if the device is not stationary.

Some embodiments of the present invention provide a fluid pulse generator for a device for measurement of an intraocular pressure, the fluid pulse generator including: a pump; a first chamber downstream of the pump and in fluid communication with the pump; a second chamber downstream of the first chamber and in fluid communication with the first chamber, wherein the second chamber includes an outlet aperture; a first valve downstream of the pump and upstream of the first chamber; and a second valve downstream of the first chamber and upstream of the second chamber.

In some embodiments, a volume of the first chamber, a volume of the second chamber, a cross section of the second valve, cross-sections of flow tubes, and a speed of opening the second valve are set to provide a desired pressure-time variation pattern a fluid pulse being puffed from the fluid pulse generator.

In some embodiments, the fluid pulse generator may include: a first pressure sensor configured to measure a fluid pressure in the first chamber; and a second pressure sensor configured to measure a fluid pressure in the second chamber.

In some embodiments, the pump, the first valve and the second valve are controllable by a controller based on readings of the first pressure sensor and the second pressure sensor.

In some embodiments, the pump, the first valve and the second valve are controllable by a controller based on a predefined pump flowrate, a predefined speed of opening and closing of first valve, and a predefined speed of opening and closing of second valve.

Some embodiments of the present invention provide a fluid pulse generator for a device for measurement of an intraocular pressure, the fluid pulse generator including: an outer body including: a cylindrical chamber configured to receive a fluid, the cylindrical chamber including a fluid outlet, and a magnetic assembly surrounding the cylindrical chamber, the magnetic assembly including a gap and configured to generate a magnetic field within at least a portion of the gap; and an inner body including: a piston tightly movable within the cylindrical chamber along a longitudinal axis thereof, and a coiled wire at a radial distance from the piston and surrounding the piston along at least a portion thereof, wherein the coiled wire is connected to the piston and is movable within the gap of the magnetic assembly of the outer body.

In some embodiments, the magnetic assembly includes: a magnetic annular structure, an annular magnet surrounding the magnetic annular structure such that a gap is formed between the magnetic annular structure and the annular magnet, wherein the annular magnet is connected at its distal end to a distal end of the magnetic annular structure, and a magnetic ring connected to a proximal end of the annular magnet structure so that a magnetic field is formed within a portion of the gap that is between the magnetic ring and the proximal end of the magnetic annular structure; wherein the outer body further includes a cover made of a non-magnetic material, and wherein the cover covers the distal end of the magnetic annular structure and includes the fluid aperture.

In some embodiments, the piston includes a cylindrical body having a closed distal end.

In some embodiments, the piston includes a piston chamber configured to receive the fluid, wherein the piston chamber is in fluid communication with the cylindrical chamber of the outer body.

In some embodiments, the piston includes: a distal circular plate at a distal end of the piston, wherein the distal circular plate is tightly movable within the cylindrical chamber of the outer body along the longitudinal axis thereof; a proximal circular plate at a proximal end of the piston; and an annular longitudinal structure connecting the distal circular plate and the proximal circular plate; wherein the piston chamber is in fluid communication with the cylindric chamber of the outer body through one or more apertures in the annular longitudinal structure, an interior of the annular longitudinal structure and an aperture in the distal circular plate.

Some embodiments of the present invention provide a fluid pulse generator including: two fluid pulse generators each as described hereinabove, wherein the two fluid pulse generators are flipped with respect to each other and are connected to each other at their base faces; and an outlet aperture in fluid communication with the fluid outlets of the two fluid pulse generators.

Some embodiments of the present invention provide a method of a non-contact measurement of the intraocular pressure (IOP) of a subject's eye, the method including: puffing a fluid pulse by the fluid pulse generator through a conduit at a cornea of the subject's eye; capturing, by at least one camera, one or more image frames, each including an image of a side view of a cornea of the subject's eye; determining, by a processing unit, one or more fluid pulse pressure values; and determining, by the processing unit, the IOP value of the subject's eye based on at least one of the one or more image frames and at least one of the one or more fluid pressure values.

Some embodiments may include: detecting, by the processing unit, based on at least one of one or more image frames, that the cornea of the subject's eye is in its first applanated state; and determining, by the processing unit, a fluid pulse pressure value at which the cornea has reached its first applanated state, wherein the determined fluid pulse pressure value is indicative of the IOP value of the subject's eye.

Some embodiments may include: determining a fluid pulse pressure value that would have caused the cornea of the subject's eye to reach its first applanated state based on a known deformation-pressure variation pattern of the subject's eye, based on a subset of the one or more image frames and based on at least one of the one or more fluid pulse pressure values, wherein the determined fluid pulse pressure value is indicative of the IOP value of the subject's eye.

Some embodiments may include determining, by the processing unit, the one or more fluid pulse pressure values based on readings of a pressure sensors configured to measure a fluid pulse pressure within the conduit.

Some embodiments may include determining, by the processing unit, the one or more fluid pulse pressure values based on a known pressure-time variation pattern of the fluid pulse and a time point at which the fluid pulse generator has puffed the fluid pulse through the conduit.

Some embodiments may include: capturing one or more image frames of the side view of the cornea by a first camera and capturing one or more image frames of the side view of the cornea by a second camera at a predefined time shift with respect to the first camera; and combining, by the processing unit, the one or more image frames captured by the first camera with the one or more image frames captured by the second camera into a single image dataset.

Some embodiments may include: capturing, by the at least one camera, the one or more image frames such that the one or more image frames further include an image of an outlet conduit; and determining, by the processing unit, a distance between the outlet conduit end and the cornea based on the one or more image frames.

Some embodiments may include: controlling, by the controller, the fluid pulse generator to puff the fluid pulse if the outlet conduit end is at a predefined distance from the cornea; and preventing, by the controller, from the fluid pulse generator to puff the fluid pulse if the outlet conduit end is not at a predefined distance from the cornea.

Some embodiments may include controlling, by the controller, a conduit positioning mechanism so as to position the outlet conduit end at the predefined distance from the cornea.

Some embodiments may include capturing, by an additional camera, one or more additional image frames each including an image of a front view of the cornea of the subject's eye.

Some embodiments may include verifying, by the processing unit, that the conduit is aligned with a center of the cornea based on the one or more additional image frames and a known position of the conduit.

Some embodiments may include: controlling, by the controller, the fluid pulse generator to puff the fluid pulse upon the verification that the conduit is aligned with the center of the cornea; and preventing, by the controller, from the fluid pulse generator to puff the fluid pulse if the conduit is not aligned with the center of the cornea.

Some embodiments may include controlling, by the controller, the conduit positioning mechanism so as to align the conduit with the center of the cornea.

Some embodiments may include: detecting, by the processing unit, a blinking of the subject's eye based on the one or more image frames; and controlling, by the controller, the fluid pulse generator to puff the fluid pulse through the conduit at a predetermined time interval after the blinking of the subject's eye ends.

Some embodiments may include controlling, by the controller, a light source to emit a light beam and aligning the light beam along the conduit axis so that the subject can align the subject's eye with the conduit axis.

Some embodiments may include controlling, by the controller, light condition control means so as to cause the subject's eye to be exposed to substantially the same lighting conditions each time one or more image frames is being captured.

Some embodiments may include determining, by the processing unit, based on readings of one or more movement sensors, at least one of: an orientation of a device in a reference coordinate system; whether a subject's right eye or a subject's left eye is being tested by a device; whether or not the device is stationary; and that the fluid pulse generator has puffed the fluid pulse.

Some embodiments may include: controlling, by the controller, the fluid pulse generator to puff the fluid pulse upon verification that the device is horizontal; and preventing, by the controller, from the fluid pulse generator to puff the fluid pulse if the device is not horizontal.

Some embodiments may include: controlling, by the controller, the fluid pulse generator to puff the fluid pulse upon verification that the device is stationary; and preventing, by the controller, from the fluid pulse generator to puff the fluid pulse if the device is not stationary.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same can be carried into effect, reference will now be made, purely by way of example, to the accompanying drawings in which like numerals designate corresponding elements or sections throughout.

In the accompanying drawings:

FIGS. 1A and 1B are schematic illustrations of a device for a non-contact measurement of an intraocular pressure (IOP) and of a subject's eye, according to some embodiments of the invention;

FIG. 1C schematically depicts a graph of a pressure-time variation pattern of a fluid pulse generatable by a fluid pulse generator of a device and illustrations of a deformation-time variation pattern of a cornea of a subject's eye in response to an application of the fluid pulse thereto, according to some embodiments of the invention;

FIG. 1D schematically depicts a graph of a fluid pulse axial velocity-time variation pattern generatable by a fluid pulse generator of a device, according to some embodiments of the invention.

FIG. 1E schematically depicts a graph of a deformation-time variation pattern of a cornea of a subject's eye in response to an application thereto of a fluid pulse generatable by a fluid pulse generator of a device, according to some embodiments of the invention;

FIG. 2 is a schematic illustration of a first embodiment of a fluid pulse generator, according to some embodiments of the invention;

FIG. 3 is a schematic illustration of a second embodiment of a fluid pulse generator, according to some embodiments of the invention;

FIG. 4A is a schematic illustration of a third embodiment of a fluid pulse generator, according to some embodiments of the invention;

FIG. 4B is a schematic illustration of a fourth embodiment of a fluid pulse generator, according to some embodiments of the invention; and

FIG. 5 is a flowchart of a method of a non-contact measurement of an intraocular pressure (IOP) of a subject's eye, according to some embodiments of the invention.

It will be appreciated that, for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the present invention are described. For purposes of explanation, specific configurations and details are set forth in order to provide a thorough understanding of the present invention. However, it will also be apparent to one skilled in the art that the present invention can be practiced without the specific details presented herein. Furthermore, well known features can have been omitted or simplified in order not to obscure the present invention. With specific reference to the drawings, it is stressed that the particulars shown are by way of example and for purposes of illustrative discussion of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention can be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that can be practiced or carried out in various ways as well as to combinations of the disclosed embodiments. Also, it is to be understood that the phraseology and terminology employed herein is for the purpose of description and should not be regarded as limiting.

Unless specifically stated otherwise, as apparent from the following discussions, it is appreciated that throughout the specification discussions utilizing terms such as “processing”, “computing”, “calculating”, “determining”, “enhancing” or the like, refer to the action and/or processes of a computer or computing system, or similar electronic computing device, that manipulates and/or transforms data represented as physical, such as electronic, quantities within the computing system's registers and/or memories into other data similarly represented as physical quantities within the computing system's memories, registers or other such information storage, transmission or display devices. Any of the disclosed modules or units can be at least partially implemented by a computer processor.

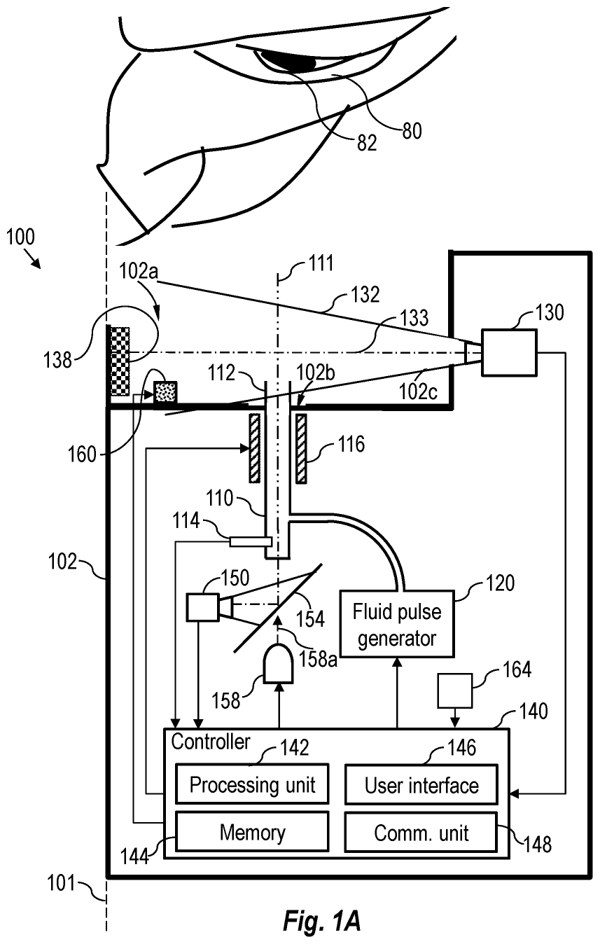

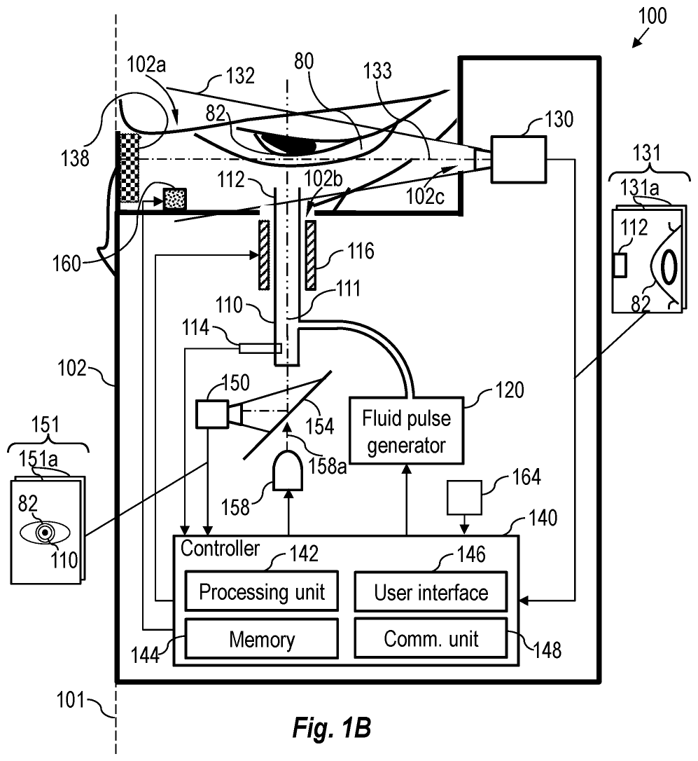

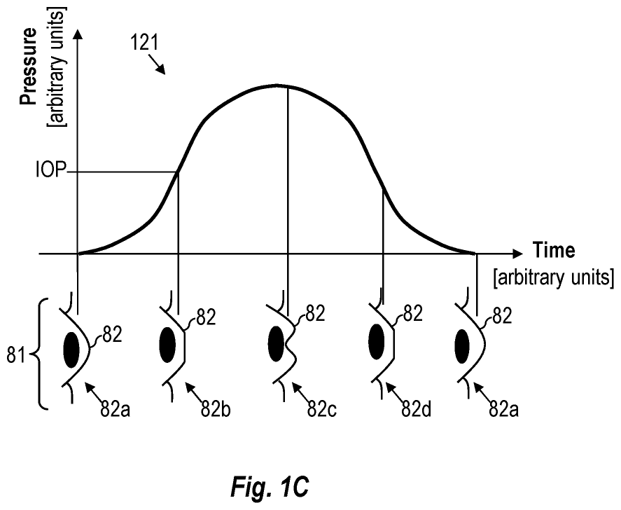

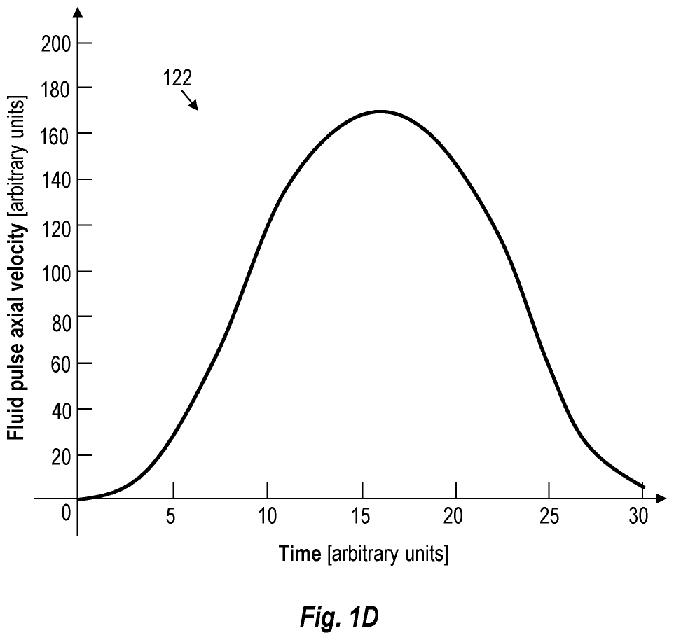

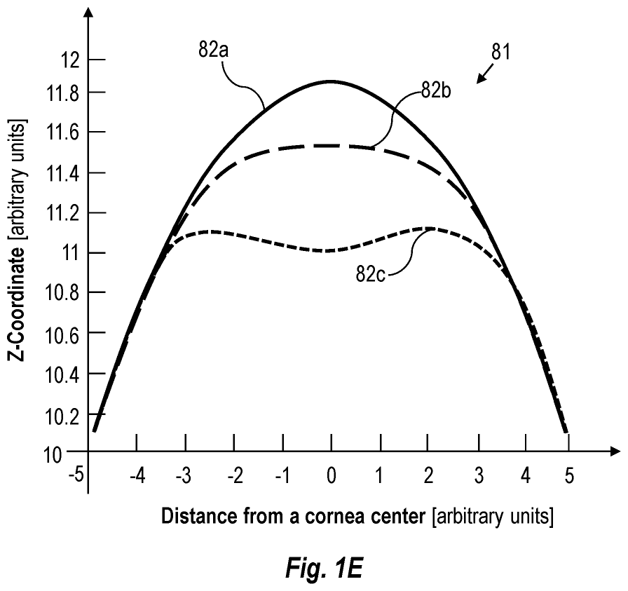

Reference is now made to FIGS. 1A and 1B, which are schematic illustrations of a device 100 for a non-contact measurement of the intraocular pressure (IOP) and of a subject's eye 80, according to some embodiments of the invention.

Reference is also made to FIG. 1C, which schematically depicts a graph of a pressure-time variation pattern 121 of a fluid pulse generatable by a fluid pulse generator 120 of a device 100 and illustrations of a deformation-time variation pattern 81 of a cornea 82 of a subject's eye 80 in response to an application of the fluid pulse thereto, according to some embodiments of the invention.

Reference is also made to FIG. 1D, which schematically depicts a graph of a fluid pulse axial velocity-time variation pattern 122 generatable by a fluid pulse generator 120 of a device 100, according to some embodiments of the invention.

Reference is also made to FIG. 1E, which schematically depicts a graph of a deformation-time variation pattern 81 of a cornea 82 of a subject's eye 80 in response to an application thereto of a fluid pulse generatable by a fluid pulse generator 120 of a device 100, according to some embodiments of the invention.

Device 100 may include a frame 102. Frame 102 may support one or more components of device 100. Frame 102 may be shaped and sized to cause a desired positioning of device 100 with respect to a subject's eye 80. For example, frame 102 may include a concave indent 102a at one of its ends and/or at one of its corners. Concave indent 102a may receive at least a portion of the subject's eye 80 and cause frame 102 to support against one or more bones of the subject's head surrounding the subject's eye 80 when device 100 is being used by the subject (e.g., as shown in FIG. 1B). In some embodiments, device 100 may include a housing surrounding at least a portion of device 100.

Device 100 may include a conduit 110. Conduit 110 may be connected, or movably connected, to frame 102. Conduit 110 may be aligned along a conduit axis 111. When device 100 is properly positioned with respect to subject's eye 80, conduit axis 111 may be radial to (or substantially radial to)/aligned with (or substantially aligned with) a center of a cornea 82 of subject's eye (e.g., as shown in FIG. 1B). In various embodiments, frame 102, or housing of device 100, may include an aperture 102b through which conduit 110 may protrude external to frame 102, or external to the housing of device 100. In some embodiments, an internal diameter of conduit 110 may be, for example, 2.4 millimeter.

Device 100 may include a fluid pulse generator 120. Fluid pulse generator 120 may be connected to frame 102. Fluid pulse generator 120 may be in fluid communication with conduit 110. Fluid pulse generator 120 may generate a fluid pulse. For example, fluid pulse generator 120 may generate a gas pulse, e.g., an air pulse. Fluid pulse generator 120 may puff the fluid pulse through conduit 110. Fluid pulse generator 120 may be configured to generate fluid pulses having different pressure-time variation patterns.

Conduit 110 may direct the fluid pulse at cornea 82 of subject's eye 80. In some embodiments, fluid pulse generator 120 may generate a fluid pulse that may deform cornea 82 from its normal convex sate 82a, through its first applanated state 82b to its concave state 82c (e.g., as shown in FIG. 1C). Cornea 82 may then revert from its concave state 82c, through its second applanated state 82d to its normal convex state 82a (e.g., as shown in FIG. 1C). A fluid pulse pressure at which cornea 82 reaches its first applanated state 82b may be indicative of an intraocular pressure (IOP) value of subject's eye 80.

One example of a pressure-time variation pattern 121 of the fluid pulse is depicted in FIG. 1C. One example of a fluid pulse axial velocity-time variation pattern 122 is depicted in FIG. 1D. For example, the fluid pulse may have a peak axis velocity of about 180 meter/second. The duration of the fluid pulse may, for example, be about 30 milliseconds. One example of a deformation-time variation pattern 81 of a cornea 82 of a subject's eye 80 is depicted in FIG. 1E.

In some embodiments, fluid pulse generator 120 may generate a fluid pulse that may slightly deform cornea 82 from its normal convex state 82a to a deformed state (e.g., that may be an intermediate state between normal convex state 82a and first applanated state 82b).

Device 100 may include at least one camera. In embodiments shown in FIGS. 1A and 1B, device 100 includes a camera 130. Camera 130 may be connected to frame 102. Camera 130 may generate an image dataset 131. Image dataset 131 may include one or more image frames 131a. Image frame(s) 131a may include an image of at least one of a side view of cornea 82 of subject's eye 80 and an outlet conduit end 112 (e.g., as shown in FIG. 1B).

In some embodiments, camera 130 may be connected to frame 102 such that a field-of-view (FOV) 132 of camera 130 is centered/located along an axis 133 that is perpendicular (or substantially perpendicular) to conduit axis 111 (e.g., as shown in FIGS. 1A and 1B). When device 100 is properly positioned with respect to subject's eye 80, axis 133 may be parallel (or substantially parallel) to a tangent of cornea 82 of subject's eye 80 (e.g., as shown in FIG. 1B).

In some embodiments, device 100 may include one or more mirrors through which camera 130 may observe at least one of outlet conduit end 112 and cornea 82, and capture image frame(s) 131a. In this case, camera 130 may be connected to frame 102 in a different manner rather than shown in FIGS. 1A and 1B. Different optical arrangements may be used so as to cause camera 130 to observe at least one of outlet conduit end 112 and cornea 82, and to capture image frame(s) 131a.

In various embodiments, frame 102, or a housing of device 100, may include an aperture 102c through which camera 130 may observe (e.g., either directly as shown in FIGS. 1A and 1B, or through one or more minors as described hereinabove) the side view of cornea 82 and outlet conduit end 112.

Device 100 may include a controller 140. Controller 140 may include a processing unit 142. Processing unit 142 may receive image frame(s) 131a from camera 130. Processing unit 142 may determine one or more fluid pulse pressure values (e.g., as described hereinbelow). Processing unit 142 may determine an intraocular pressure (IOP) value of subject's eye 80 based on at least one of image frame(s) 131a and at least one of the determined fluid pulse pressure value(s).

In some embodiments, processing unit 142 may detect, based on at least one of image frame(s) 131a, that cornea 82 of subject's eye 80 is in its first applanated state 82b and may determine a fluid pulse pressure value at which cornea 82 has reached its first applanated state 82b (e.g., as described hereinbelow). The fluid pulse pressure value at which cornea 82 has reached its first applanated state 82b may be indicative of the IOP value of subject's eye 80.

In some embodiments, controller 140 may include a memory 144. Processing unit 142 may, for example, save the determined IOP value of subject's eye in memory 144. The determined IOP value may be, for example, accompanied with a metadata when saved into memory 144. The metadata may, for example, include a date and a time at which the IOP value has been determined.

In some embodiments, controller 140 may include a user interface 146. Controller 140 may, for example, notify the subject concerning the determined IOP value via user interface 146. In various embodiments, user interface 146 may generate visual and/or voice notifications. In some embodiments, user interface 146 may receive voice instructions from the subject and controller 140 may control device 100 based on the voice instructions thereof.

In some embodiments, device 100 may include one or more pressure sensors 114. Pressure sensor(s) 114 may measure a fluid pulse pressure. For example, pressure sensor(s) 114 may measure the fluid pulse pressure in conduit 110. Processing unit 142 may determine the fluid pulse pressure values (e.g., the fluid pulse pressure value at which cornea 82 has reached its first applanated state 82b) based on readings of pressure sensor(s) 114.

In some embodiments, a pressure-time variation pattern of the fluid pulse may be known. The pressure-time variation pattern of the fluid pulse may be predefined by, for example, a structure and operational parameters of fluid pulse generator 120. In this case, processing unit 142 may determine the fluid pulse pressure values (e.g., the fluid pulse pressure value at which cornea 82 has reached its first applanated state 82b) based on the known/predefined pressure-time variation pattern of the fluid pulse and a time point at which the fluid pulse has been puffed by fluid pulse generator 120 through conduit 110. In some embodiments, the known/predefined pressure-time variation pattern may be saved in memory 144.

In some embodiments, processing unit 142 may determine the time point at which the fluid pulse has been puffed by fluid pulse generator 120 based on readings of pressure sensor(s) 114. In this case, pressure sensor(s) 114 may be, for example, a low-end and cheap sensor(s) having relatively low sampling rate. In some embodiments, processing unit 142 may receive the time point at which the fluid pulse has been puffed by fluid pulse generator 120 from controller 140. In some embodiments, processing unit 142 may determine the time point at which the fluid pulse has been puffed by fluid pulse generator 120 based on readings of one or more movement sensors 164 (e.g., as described hereinbelow).

In some embodiments, camera 130 may be a high-speed camera. For example, camera 130 may be capable of capturing 500 to 5,000 image frames per second, for example 1,000 image frames per second. In some embodiments, camera 130 may be a line-scan camera. In some embodiments, camera 130 is a surface camera.

In some embodiments, device 100 may include two cameras, e.g., a first camera and a second camera, configured to capture image frames including an image of at least one of the side view of cornea 82 and outlet conduit end 112 (e.g., such as image frame(s) 131a). In various embodiments, the first camera and/or the second camera may be line-scan cameras. In various embodiments, the first camera and/or the second camera may be surface cameras. The image frame rate of each of the first camera and the second camera may be half of an image frame rate that may be required when using a single camera, e.g., such as camera 130. The first camera and the second camera may capture the respective image frames at a predefined time shift with respect to each other. Processing unit 142 may combine the image frames being captured by the first camera and the image frames being captured by the second camera into a single combined image dataset that may be similar (or substantially similar) to an image dataset that may be generated by a single camera, e.g., such as image dataset 131 generated by camera 130.

For example, instead of a single camera having an image frame rate of 1,000 image frames per second, a first camera and a second camera each having an image frame rate of 500 image frames per second may be used. The first camera and second camera may capture the respective image frames at a predefined time shift of, e.g., 1 millisecond with respect to each other. The image frames being captured by the first camera and the image frames being captured by the second camera at the image frame rate of 500 image frames per second and at the time shift of 1 millisecond with respect to each other may be combined by processing unit 142 into a single combined image dataset that may be similar to an image dataset that may be generated by a single camera having an image frame rate of 1,000 image frames per second.

Such relatively low-speed first and second cameras may be significantly cheaper than a single high-speed camera, while operation of the first and second cameras at the predefined time shift with respect to each other may yield the same (or substantially the same) image dataset as may be generated by the single high-speed camera.

In some embodiments, processing unit 142 may determine a fluid pulse pressure value that would have cause cornea 82 of subject's eye 80 to reach its first applanated state 82b based on a known deformation-pressure variation pattern of the subject's eye, a subset of image frames 131a and at least one of the fluid pulse pressure value(s). The determined fluid pulse pressure value may be indicative of the IOP value of subject's eye. The deformation-pressure variation pattern of cornea 82 may be known or predefined. For example, the deformation-pressure variation pattern of cornea 82 may be a subject-specific pattern derived based multiple previous measurements made for the subject. In another example, the deformation-pressure variation pattern of cornea 82 of the subject may be derived based premeasured deformation-pressure patterns of multiple individuals having common characteristics. In some embodiments, the known/predefined deformation-pressure variation pattern of cornea 82 may be stored in memory 144. The subset of image frames 131a may, for example, include few image frames taken during initial stages of deformation of cornea 82 due to application of the fluid pulse thereto. The fluid pressure value(s) may be determined either based on readings of pressure sensor(s) 114 or based on the known/predefined pressure-time variation pattern of the fluid pulse (e.g., as described hereinabove). In this case, fluid pulse generator 120 may be set to generate a fluid pulse having relatively low peak pressure value that does not cause cornea 82 to deform to its first applanated state 82b. Since deformation-pressure variation pattern of cornea 82 of subject's eye 80 may be known/predefined, it may be sufficient to capture few image frames of slight deformation of cornea 82 due to application of the fluid pulse thereto and the IOP value of subject's eye 80 may then be determined based on the known/premeasured deformation-pressure variation pattern of cornea 82. Using fluid pulses having relatively low peak pressure value may, for example, cause less inconvenience to the subject undergoing the test. Furthermore, since it may be sufficient to capture few image frames of slight deformation of cornea 82, a relatively cheap camera having relatively low image frame rate may be used instead of a high-speed camera.

In some embodiments, device 100 may include a visual reference marker 138. In some embodiments, visual reference marker 138 may include at least one of a visual length marker, a visual position marker and a visual color marker. Visual reference marker 138 may be connected to frame 102. For example, visual marker 138 may be connected to frame 102 within concave indent 102a thereof. Visual reference marker 138 may be opposite to camera 130 along axis 133 and/or opposite to an aperture 102c through which camera 130 may observe cornea 82 and outlet conduit end 112 so that image frame(s) 131a being captured by camera 130 may also include an image of at least a portion of visual reference marker 138. Processing unit 142 may, for example, scale image frame(s) 131a based on the image of at least one of the visual length marker and the visual position marker. The visual color marker may, for example, enhance a contrast of image frame(s) 131a being captured so as to improve the processing of image frame(s) 131a.

In some embodiments, device 100 may include a camera 150. Camera 150 may be connected to frame 102. Camera 150 may generate an image dataset 151. Image dataset 151 may include one or more image frames 151a. Image frame(s) 151a may include an image of at least one of a front view of cornea 82 of subject's eye 80 and conduit 110 (e.g., as shown in FIG. 1B).

In some embodiments, camera 150 may observe the front view of cornea 82 of the subject's eye 80 and conduit 110 through one or more minors or through one or more half-minors (e.g., through a half minor 154 as shown in FIGS. 1A and 1B). In some other embodiments, camera 150 may be connected to frame 102 such that a field of view of camera 150 is centered/located along an axis that is parallel (or substantially parallel) to conduit axis 111 (and thus may directly observe cornea 82 and/or conduit 110). Different optical arrangement may be used to cause second camera 150 to observe the front view of cornea 82 of subject's eye 80 and conduit 110, and to capture image frame(s) 151a.

In some embodiments, processing unit 142 may receive image frame(s) 151a. Processing unit 142 may verify that conduit 110 is radial to/aligned with the center of cornea 82 of subject's eye 80 based on image frame(s) 151a. For example, processing unit 142 may verify that conduit 110 is radial to/aligned with the center of cornea 82 of subject's eye 80 based on image frame(s) 151a including an image of cornea 82 and a known position of conduit 110 with respect to frame 102. The position of conduit 110 may be derived from image frame(s) 151a (if image frame(s) contain the image of conduit 110) or may be received from controller 140 that may store the position of conduit 110.

In some embodiments, device 100 may include a conduit positioning mechanism 116.

Conduit positioning mechanism 116 may move conduit 110 with respect to frame 102 in a controlled manner. In some embodiments, conduit positioning mechanism 116 may move conduit 110 along a conduit axis 111. In some embodiments, conduit positioning mechanism 116 may move conduit 110 in one or more directions that are perpendicular to conduit axis 111. In some embodiments, conduit positioning mechanism 116 may rotate conduit 110 about at least one axis that is perpendicular to conduit axis 111. Conduit positioning mechanism 116 may, for example, include, for each axis of movement, an electric motor with or without feedback sensor. For example, each axis of movement may be a closed loop servo system.

Controller 140 may control conduit positioning mechanism 116 in order to position conduit 110 in a proper position with respect to cornea 82 of subject's eye 80. In various embodiments, controller 140 may control conduit positioning mechanism 116 based on image frame(s) 131a from camera 130 and/or based on image frame(s) 151 from camera 150 being processed by processing unit 142.

In some embodiments, processing unit 142 may determine a distance between outlet conduit end 112 and cornea 82 based on image frame(s) 131a from camera 130. Processing unit 142 may, for example, track the distance between outlet conduit end 112 and cornea 82. For example, processing unit 142 may track the distance in real-time, or substantially in real-time. Controller 140 further may control conduit positioning mechanism 116 to move conduit 110 along conduit axis 111 so as to position outlet conduit end 112 at a predefined distance from cornea 82. The predefined distance may be within a range of 5 to 20 millimeters, for example, 11 millimeters.

In some embodiments, controller 140 may control conduit positioning mechanism 116 to move conduit 110 away from subject's eye 80 if the distance between outlet conduit end 112 and cornea 82 is below a specified distance threshold. In some embodiments, controller 140 may control fluid pulse generator 120 to puff the fluid pulse through conduit 110 upon verification that outlet conduit end 112 is at the predefined distance from cornea 82. In some embodiments, controller 140 may prevent fluid pulse generator 120 from puffing the fluid pulse through conduit 110 if the distance of outlet conduit end 112 from cornea 82 is not the predefined distance.

In some embodiments, processing unit 142 may determine that conduit 110 is radial to/aligned with the center of cornea 82 of subject's eye 80 based on image frame(s) 151a from camera 150. In some embodiments, controller 140 may control fluid pulse generator 120 to puff the fluid pulse through conduit 110 upon verification that conduit 110 is radial to/aligned with the center of cornea 82 of subject's eye 80. In some embodiments, controller 140 may prevent fluid pulse generator 120 from puffing the fluid pulse through conduit 110 if conduit 110 is not radial to/aligned with the center of cornea 82 of subject's eye 80.

In some embodiments, device 100 may include a light source 158. Light source 158 may emit a light beam 158a that may be aligned along conduit axis 111. For example, light beam 158 may be generated by an image projector. Light beam 158a may be used by the subject as a reference point to align cornea 82 of its eye 80 with conduit axis 111. In some embodiments, light source 158 may be connected to frame 102 along conduit axis 111, and light beam 158a from light source 158 may pass towards subject's eye 80 through half-minor 154 (e.g., as shown in FIGS. 1A and 1B). In some other embodiments, light beam 158a from light source 158 may be aligned along conduit axis 111 using one or more minors. In this case, light source 158 may be connected to frame 102 in a different manner rather than shown in FIGS. 1A and 1B.

In some embodiments, processing unit 142 may detect a blinking of subject's eye 80 based on at least one of image frame(s) 131a from camera 130 and image frame(s) 151a from camera 150. Controller 140 may control fluid pulse generator 120 to puff the fluid pulse through conduit 110 at a predetermined time interval after the blinking of subject's eye 80 ends.

In some embodiments, device 100 may include light condition control means 160. Light condition control means 160 may cause subject's eye 80 to be exposed to the same lighting conditions (or substantially the same lighting conditions) each time device 100 is used. For example, light condition control means 160 may include at least one of: a controllable light source and a controllable shadowing means. In some embodiments, controller 140 may control light condition control means 160 based on at least one of image frame(s) 131a from camera 130 and image frame(s) 151a from camera 150 being processed by processing unit 142. In various embodiments, controller 140 may adjust image frame(s) 151a and/or image frame(s) 131a received from cameras 150 and 130, respectively, based on the light condition data received from light condition control means 160.

In some embodiments, device 100 may include one or more movement sensors 164. Movement sensor(s) 164 may be connected to frame 102. Movement sensor(s) 164 may, for example, include one or more accelerometers, one or more gyroscopes, etc.

In some embodiments, processing unit 142 may determine an orientation of device 100 in a reference coordinate system (e.g., global coordinate system) based on readings of inertial/accelerometer sensor(s) 164. For example, processing unit 142 may determine whether or not device 100 is horizontal based on readings of inertial/accelerometer sensor(s) 164. In some embodiments, controller 140 may control fluid pulse generator 120 to puff the fluid pulse through conduit 110 upon verification that device 100 is horizontal. In some embodiments, controller 140 may prevent fluid pulse generator 120 from puffing the fluid pulse through conduit 110 if device 100 is not horizontal. Controller 140 may, for example, instruct the subject (e.g., by visual and/or voice notification via user interface 146) how to move and orient device 100 to properly position it with respect to subject's eye 80.

In another example, processing unit 142 may determine whether a subject's right eye or a subject's left eye is being tested by device 100 based on readings of inertial/accelerometer sensor(s) 164. For example, in order to test the subject's right eye or the subject's left eye, device 100 may be flipped between its first flipped position and a second flipped position about an axis 101 that is parallel (or substantially parallel) to conduit axis 111. Processing unit 142 may determine whether device 100 is in its first flipped position or in its second flipped position based on readings of inertial/accelerometer sensor(s) 164 (e.g., gyroscopes) and may thus determine which of the subject's right eye or the subject's left eye is being tested by device 100. In some embodiments, the metadata accompanying the determined IOP value being saved in memory 144 may include information indicating whether the IOP value has been determined for the subject's left or right eye.

In some embodiments, processing unit 142 may determine whether or not device 100 is stationary based on readings of movement sensor(s) 164. In some embodiments, controller 140 may control fluid pulse generator 120 to puff the fluid pulse through conduit 110 upon verification that device 100 is stationary. In some embodiments, controller 140 may prevent fluid pulse generator 120 from puffing the fluid pulse through conduit 110 if device 100 is not stationary.

In some embodiments, processing unit 142 may detect, based on readings of movement/accelerometer sensor(s) 164, that fluid pulse generator 120 has puffed the fluid pulse through conduit 110. For example, movement/accelerometer sensor(s) 164 may detect an impulse due to the fluid pulse being puffed by fluid pulse generator 120 and/or due to operation of fluid pulse generator 120.

In some embodiments, processing unit 142 may determine one or more cornea parameters of cornea 82 of subject's eye 80 based on at least one of image frame(s) 131a from camera 130 and image frame(s) 151a from camera 150. For example, the cornea parameter(s) may include a thickness of cornea 82, a shape of surface of cornea 82, hysteresis of cornea 82, contour of cornea 82, etc. In some embodiments, processing unit 142 may save the determined cornea parameter(s) in memory 144.

In some embodiments, controller 140 may include a communication unit 148. Communication unit 148 may, for example, send information (e.g., information stored in memory 144, determined IOP values, etc.) to a third authorized party (e.g., an appointed doctor, etc.). Communication unit 148 may, for example, send information to a remote server (e.g., cloud, etc.). Communication unit 148 may, for example, communicate with a mobile application running on a smartphone of the subject.

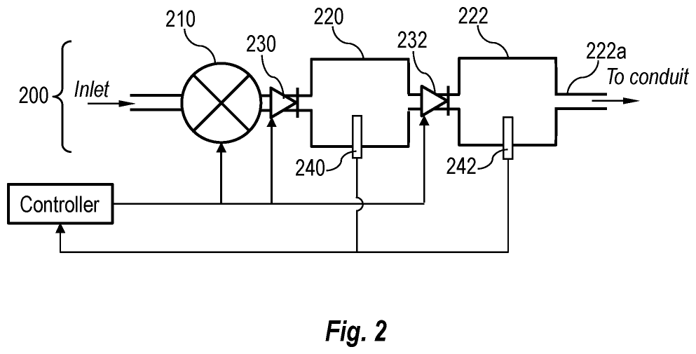

Device 100 may include various embodiments of fluid pulse generator 120. For example, fluid pulse generator 120 may include bellows, linear or rotational solenoids, linear motors, etc. Some embodiments of fluid pulse generator 120 are described below with respect to FIGS. 2, 3, 4A and 4B.

In some embodiments, device 100 may include fasteners that may fasten device 100 to a subject's head. In some embodiments, device 100 may be a hand-held device. In some embodiments, device 100 may be portable.

Utilization of device 100 does not require any special technical skills. Accordingly, the measurement of the IOP may be performed by the subject itself, for example, at subject's home, without a need in visiting an ophthalmologist or a skilled technician. Device 100 may enable the subject to measure the IOP on a daily basis, or several times a day, according to ophthalmologist's instructions and subject's condition, while reducing the frequency of clinic IOP measurements to minimum. Device 100 may, for example, recommend that the subject get medical advice if the determined IOP value exceeds some predetermine IOP value levels. Device 100 may, for example, recommend ignoring the determined IOP values if the determined IOP values are not within a specified range of IOP values.

Reference is now made to FIG. 2, which is a schematic illustration of a first embodiment of a fluid pulse generator 200, according to some embodiments of the invention.

Fluid pulse generator 200 may include a pump 210, a first chamber 220 and a second chamber 222. First chamber 220 may be downstream of pump 210 and may be in fluid communication with pump 210. Pump 210 may pump a fluid (e.g., a gas such as air) into first chamber 220. Second chamber 222 may be downstream of first chamber 220 and may be in fluid communication with first chamber 220. Second chamber 222 may include an outlet aperture 222a. Outlet aperture 222a may be connectable to a conduit (e.g., such as conduit 110 described hereinabove).

Fluid pulse generator 200 may include a first valve 230 between pump 210 and first chamber 220. Fluid pulse generator 200 may include a second valve 232 between first chamber 220 and second chamber 222.

In some embodiments, pump 210 may be a diaphragm pump that includes first valve 230.

In some embodiments, fluid pulse generator 200 may include a first pressure sensor 240 to measure a fluid pressure in first chamber 220. In some embodiments, fluid pulse generator 200 may include a second pressure sensor 242 to measure a fluid pressure in second chamber 222.

In some embodiments, a volume of first chamber 220, a volume of second chamber 222, a speed of opening second valve 232, a cross section of second valve 232, cross-sections of flow tubes may be predefined to provide a desired pressure-time variation pattern to the fluid pulse.

In some embodiments, a controller (e.g., such as controller 140 described hereinabove) may control pump 210, first valve 230 and second valve 232 based on readings of first pressure sensor 240 and readings of second pressure sensor 242. In some embodiments, the controller may control pump 210, first valve 230 and second valve 232 based on a predefined pump flowrate, a predefined speed of opening and closing of first valve 230, a predefined speed of opening and closing of second valve 232.

Fluid pulse generator 200 has a minimal number of moving mechanical components (e.g., pump 210, first valve 230 and second valve 232) and has no heavy fast-moving mechanical components. Thus, the operation of fluid pulse generator 200 does not produce a significant impulse.

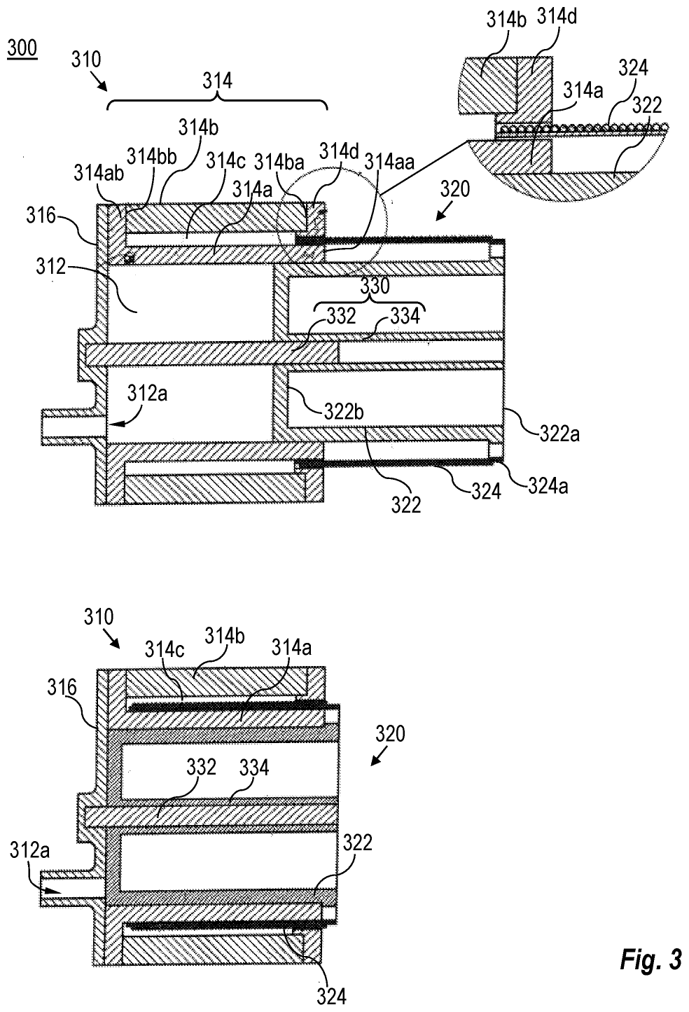

Reducing an impulse generated by the fluid pulse generator 200 may be important in order to prevent a motion of an IOP measurement device (e.g., device 100 described hereinabove) upon puffing of the fluid pulse at the subject's eye. Reference is now made to FIG. 3, which is a schematic illustration of a second embodiment of a fluid pulse generator 300, according to some embodiments of the invention.

FIG. 3 shows a schematic longitudinal cut view of fluid pulse generator 300.

Fluid pulse generator 300 may include an outer body 310 and an inner body 320.

Outer body 310 may include a cylindrical chamber 312. Cylindrical chamber 312 may receive a fluid (e.g., a gas, such as air). Cylindrical chamber 312 may include a fluid aperture 312a. For example, the fluid may be injected from/into cylindrical chamber 312 through fluid aperture 312a.

Outer annular body 310 may include a magnetic assembly 314. Magnetic assembly 314 may surround cylindrical chamber 312.

In some embodiments, magnetic assembly 314 may include a magnetic annular structure 314a having a proximal end 314aa and a distal end 314ab, an annular magnet 314b surrounding magnetic annular structure 314a along at least a portion of a length thereof and having a proximal end 314ba and a distal end 314bb, a gap 314c between magnetic annular structure 314a and annular magnet 314b, and a magnetic ring 314d. Magnetic ring 314d may be connected to proximal end 314ba of annular magnet 314b. Annular magnet 314b may be connected at its distal end 314bb to distal end 314ab of magnetic annular structure 314a. This may, for example, cause magnetic ring 314d and magnetic annular structure 314a to have opposite magnetic poles. This may generate a magnetic field within gap 314c in a region that is between magnetic ring 314d and proximal end 314aa of magnetic annular structure 314a.

Magnetic annular structure 314a and/or magnetic ring 314d may be made of a magnetic material having relatively high magnetic permeability that may guide magnetic field. For example, magnetic annular structure 314a and/or magnetic ring 314d may be made of Metglas, Permalloy, Mu-metal, soft iron, cobalt iron, etc. Annular magnet 314b may be made of, for example, a permanent magnet.

In some embodiments, outer body 310 may include a cover 316 that may cover distal end 314ab of magnetic annular structure 314a. Cover 316 may be made of, for example, a non-magnetic material. For example, cover 316 may be made of a plastic. In some embodiments, fluid aperture 312a may be made in cover 316.

Outer body 310 may be stationary. For example, outer body 310 may be fixed to a frame of a device such as frame 102 of device 100 as described hereinabove.

Inner body 320 may include a piston 322. In embodiments shown in FIG. 3, piston 322 may be a cylindrical body (e.g., hollow cylindrical body) having a closed distal end 322b. Piston 322 may be tightly movable within cylindrical chamber 312 of outer body 310 along a longitudinal axis thereof.

Inner body 320 may include a coiled wire 324. Coiled wire 324 may surround piston 322 along at least a portion of a length thereof. Coiled wire 324 may be at a radial distance from piston 322. Coiled wire 324 may be connected to piston 322. For example, coiled wire 324 may be connected at its proximal end 324a to a proximal end 322a of piston 322. Coiled wire 324 may be movable within gap 314c of magnetic assembly 314 of outer body 310.

Coiled wire 324 may be connected to a controller (e.g., such as controller 140 described hereinabove). The controller may controllably supply an electrical current to coiled wire 324. Electrical current flowing through coiled wire 324 located in the magnetic field within gap 314c, may generate a pushing force that may push inner body 320 into outer body 310, and puff the fluid from cylindrical chamber 312 through fluid aperture 312a.

Electrical current flowing through coiled wire 324 in a second direction that is opposite to the first direction may generate a pulling force (that is opposite to the pushing force) that may pull inner body 320 from outer body 310 and suck a fluid into cylindrical chamber 312.

In some embodiments, piston may be made of, for example, a plastic material. In this manner, inner body 320 may have relatively low weight. Accordingly, inner body 320 may generate relatively low impulse when being moved.

In some embodiments, fluid pulse generator 300 may include inner body guiding means 330 to guide the motion of inner body 320 within outer body 310. Inner body guiding means 330 may include a rail 332 positioned within cylindrical chamber 312 of outer body 310 and aligned along an axis that is parallel to the longitudinal axis of outer body 310. Inner body guiding means 330 may include a conduit 334 made within piston 322 of inner body 310. Conduit 334 may receive rail 332 so that inner body 320 may slide along rail 332 with respect to outer body 310.

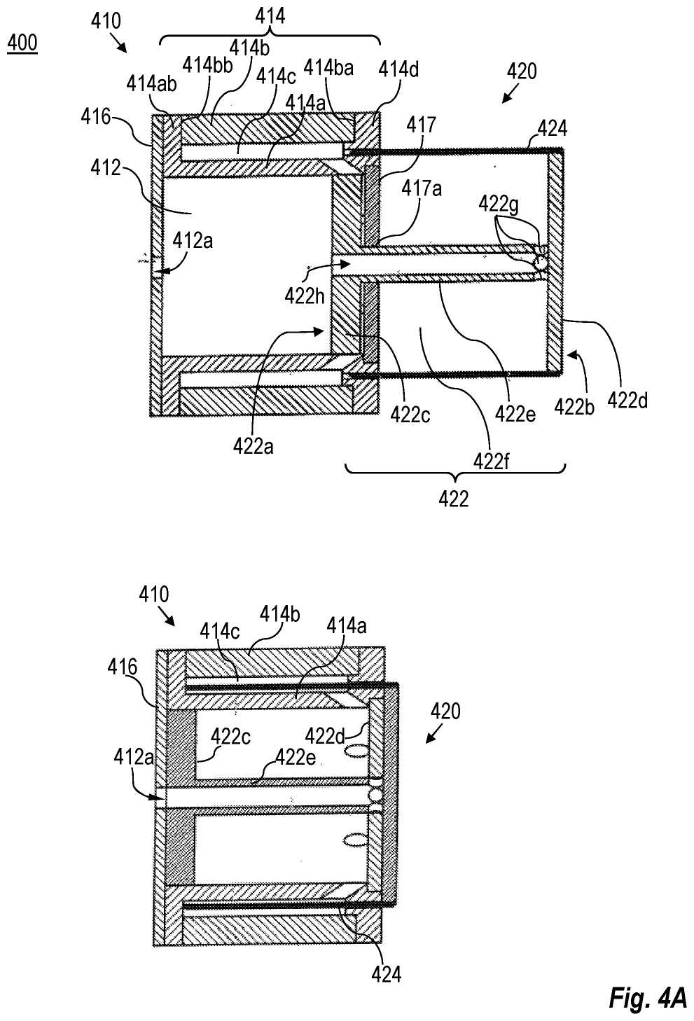

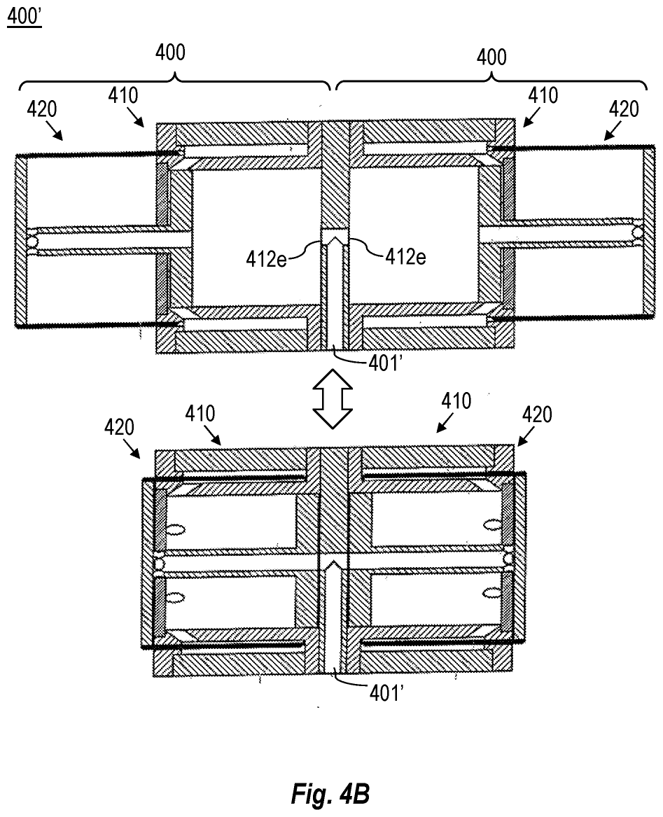

Reference is now made to FIG. 4A, which is a schematic illustration of a third embodiment of a fluid pulse generator 400, according to some embodiments of the invention.

FIG. 4A shows a schematic longitudinal cut view of fluid pulse generator 400.

Fluid pulse generator 400 may include an outer body 410 and an inner body 420.

Outer body 410 may include a cylindrical chamber 412. Cylindrical chamber 412 may receive a fluid (e.g., a gas, such as air). Cylindrical chamber 412 may include a fluid aperture 412a. For example, the fluid may be injected from/into cylindrical chamber 412 through fluid aperture 412a.

Outer body 410 may include a magnetic assembly 414. Magnetic assembly 414 may surround cylindrical chamber 412.

In some embodiments, magnetic assembly 414 may include a magnetic annular structure 414a having a proximal end 414aa and a distal end 414ab, an annular magnet 414b surrounding magnetic annular structure 414a along at least a portion of a length thereof and having a proximal end 414ba and a distal end 414bb, a gap 414c between magnetic annular structure 414a and annular magnet 414b, and a magnetic ring 414d. Magnetic ring 414d may be connected to proximal end 414ba of annular magnet 414b. Annular magnet 414b may be connected at its distal end 414bb to distal end 414ab of magnetic annular structure 414a. This may, for example, cause magnetic ring 414d and magnetic annular structure 414a to have opposite magnetic poles. This may generate a magnetic field within gap 414c in a region that is between magnetic ring 414d and proximal end 414aa of magnetic annular structure 414a.

Magnetic annular structure 414a and/or magnetic ring 414d may be made of a magnetic material having relatively high magnetic permeability that may guide magnetic field. For example, magnetic annular structure 414a and/or magnetic ring 414d may be made of Metglas, Permalloy, Mu-metal, soft iron, cobalt iron, etc. Annular magnet 414b may be made of, for example, a permanent magnet.

In some embodiments, outer body 410 may include a cover 416 that may cover distal end 414ab of magnetic annular structure 414a. Cover 416 may be made of, for example, a non-magnetic material. For example, cover 416 may be made of a plastic. In some embodiments, fluid aperture 412a may be made in cover 416.

Outer body 410 may be stationary. For example, outer body 410 may be fixed to a frame of a device such as frame 102 of device 100 as described hereinabove.

Inner body 420 may include a piston 422 having a proximal end 422a and a distal end 422b. In embodiments shown in FIG. 4A, piston 422 may include a distal circular plate 422c at its distal end 422b. Distal circular plate 422c may be tightly movable within cylindrical chamber 412 of outer body 410. Piston 422 may include a proximal circular plate 422d at its proximal end 422a. Proximal circular plate 422d may be connected to distal end 422b of piston 422 by an annular longitudinal structure 422e. Proximal circular plate 422d may have a greater diameter than distal circular plate 422c. Piston 422 may include a piston chamber 422f. Piston chamber 422f may be in fluid communication with cylindrical chamber 412 of outer body 410 through apertures 422g of annular longitudinal structure 422e, an interior of annular longitudinal structure 422e and an aperture 422h in distal circular plate 422c.

In some embodiments, outer body 410 may include a proximal cover 417. Proximal cover 417 may cover proximal end 414aa of magnetic annular structure 414a. Proximal cover 417 may include an aperture 417a through which annular longitudinal structure 422e of piston 422 may pass.

Inner body 420 may include a coiled wire 424. Coiled wire 424 may surround piston 422 along at least a portion of a length thereof. Coiled wire 424 may be at a radial distance from at least a portion of piston 422. Coiled wire 424 may be connected at its proximal end 424a to proximal circular plate 422d of piston 422. Coiled wire 424 may be movable within gap 414c of magnetic assembly 414 of outer body 410.

Coiled wire 424 may be connected to a controller (e.g., such as controller 140 described hereinabove). The controller may controllably supply an electrical current to coiled wire 424. Electrical current flowing through coiled wire 424 located in the magnetic field within gap 414c, may generate a pushing force that may push inner body 420 into outer body 410, and puff the fluid from cylindrical chamber 412 through fluid aperture 412a.

Electrical current flowing through coiled wire 424 in a second direction that is opposite to the first direction may generate a pulling force (that is opposite to the pushing force) that may pull inner body 420 from outer body 410 and suck a fluid into cylindrical chamber 412.

In some embodiments, piston may be made of, for example, a plastic material. In this manner, inner body 420 may have relatively low weight. Accordingly, inner body 420 may generate relatively low impulse when being moved.

Reference is now made to FIG. 4B, which is a schematic illustration of a fourth embodiment of a fluid pulse generator 400′, according to some embodiments of the invention.

FIG. 4B shows a schematic longitudinal cut view of fluid pulse generator 400′.

Fluid pulse generator 400′ may include two fluid pulse generators 400, flipped with respect to each other and connected to each other at their base faces. Fluid pulse generator 400′ may include an outlet aperture 401′ in fluid communication with fluid outlets 412e of fluid pulse generators 400. Two fluid pulse generators 400 may be controlled by the controlled to operate simultaneously so as no impulse (or substantially no impulse) is generated by fluid pulse generator 400′.

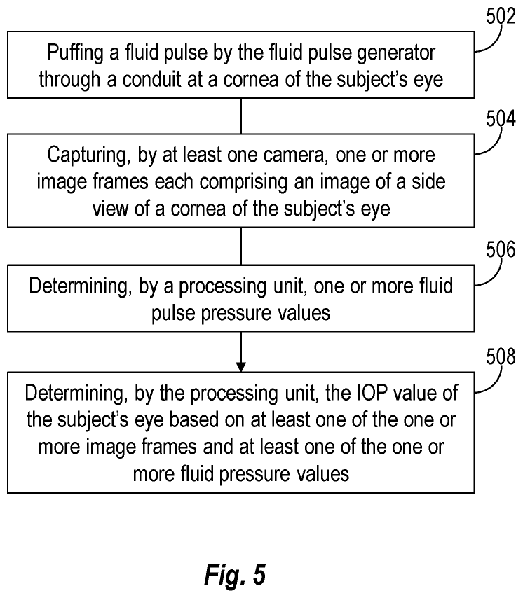

Reference is now made to FIG. 5, which is a flowchart of a method of a non-contact measurement of an intraocular pressure (IOP) of a subject's eye, according to some embodiments of the invention.

The method may be implemented by device 100, which may be configured to implement the method.

The method may include puffing 502 a fluid pulse by the fluid pulse generator through a conduit at a cornea of the subject's eye, for example, as described hereinabove.

The method may include capturing 504, by at least one camera, one or more image frames each comprising an image of a side view of a cornea of the subject's eye, for example, as described hereinabove.

The method may include determining 506, by a processing unit, one or more fluid pulse pressure values, for example, based on readings of one or more pressure sensors or based on a known/predefined pressure-time variation pattern of the fluid pulse, as described hereinabove.

The method may include determining 508, by the processing unit, the IOP value of the subject's eye based on at least one of the one or more image frames and at least one of the one or more fluid pressure values, for example, as described hereinabove.

Some embodiments may include detecting, by the processing unit, based on at least one of one or more image frames, that the cornea of the subject's eye is in its first applanated state, for example, as described hereinabove.

Some embodiments may include determining, by the processing unit, a fluid pulse pressure value at which the cornea has reached its first applanated state, wherein the determined fluid pulse pressure value is indicative of the IOP value of the subject's eye, for example, as described hereinabove.

Some embodiments may include determining, by the processing unit, the one or more fluid pulse pressure values based on readings of a pressure sensors configured to measure a fluid pulse pressure within the conduit, for example, as described hereinabove.

Some embodiments may include determining, by the processing unit, the one or more fluid pulse pressure values based on a known pressure-time variation pattern of the fluid pulse and a time point at which the fluid pulse generator has puffed the fluid pulse through the conduit, for example, as described hereinabove.

Some embodiments may include determining, by the processing unit, the time point at which the fluid pulse generator has puffed the fluid pulse based on readings of the one or more pressure sensors, for example, as described hereinabove. Some embodiments may include determining, by the processing unit, the time point at which the fluid pulse generator has puffed the fluid pulse based on readings of one or more movement/accelerometer sensors, for example, as described hereinabove. Some embodiments may include receiving, by the processing unit, the time point at which the fluid pulse generator has puffed the fluid pulse from a controller, for example, as described hereinabove.

Some embodiments may include determining, by the processing unit, a fluid pulse pressure value that would have cause the cornea of the subject's eye to reach its first applanated state based on a known deformation-pressure variation pattern of the cornea, a subset of the one or more image frames, and at least one of the one or more determined fluid pulse pressure values, for example, as described hereinabove.

Some embodiments may include capturing one or more image frames of the side view of the cornea by a first camera and capturing one or more image frames of the side view of the cornea by a second camera at a predefined time shift with respect to the first camera, for example, as described hereinabove.

Some embodiments may include combining, by the processing unit, the one or more image frames captured by the first camera with the one or more image frames captured by the second camera into a single image dataset, for example, as described hereinabove.

Some embodiments may include capturing, by the at least one camera, the one or more image frames such that the one or more image frames further comprise an image of an outlet conduit, for example, as described hereinabove.

Some embodiments may include determining, by the processing unit, a distance between the outlet conduit end and the cornea based on the one or more image frames, for example, as described hereinabove.

Some embodiments may include controlling, by the controller, the fluid pulse generator to puff the fluid pulse if the outlet conduit end is at a predefined distance from the cornea, for example, as described hereinabove.

Some embodiments may include preventing, by the controller, the fluid pulse generator from puffing the fluid pulse if the outlet conduit end is not at a predefined distance from the cornea, for example, as described hereinabove.

Some embodiments may include controlling, by the controller, a conduit positioning mechanism so as to position the outlet conduit end at the predefined distance from the cornea, for example, as described hereinabove.

Some embodiments may include capturing, by an additional camera, one or more additional image frames, each comprising an image of a front view of the cornea of the subject's eye and the conduit, for example, as described hereinabove.

Some embodiments may include verifying, by the processing unit, that the conduit is aligned with a center of the cornea based on the one or more additional image frames, for example, as described hereinabove.

Some embodiments may include controlling, by the controller, the fluid pulse generator to puff the fluid pulse upon the verification that the conduit is aligned with the center of the cornea, for example, as described hereinabove.

Some embodiments may include preventing, by the controller, the fluid pulse generator from puffing the fluid pulse if the conduit is not aligned with the center of the cornea, for example, as described hereinabove.

Some embodiments may include controlling, by the controller, the conduit positioning mechanism so as to align the conduit with the center of the cornea, for example, as described hereinabove.

Some embodiments may include detecting, by the processing unit, a blinking of the subject's eye based on the one or more image frames, for example, as described hereinabove.

Some embodiments may include controlling, by the controller, the fluid pulse generator to puff the fluid pulse through the conduit at a predetermined time interval after the blinking of the subject's eye ends, for example, as described hereinabove.

Some embodiments may include controlling, by the controller, a light source to emit a light beam and aligning the light beam along the conduit axis so as the subject can align the subject's eye with the conduit axis, for example, as described hereinabove.

Some embodiments may include determining, by the processing unit, based on readings of one or more movement/accelerometer sensors, an orientation of the device in a reference coordinate system, for example, as described hereinabove.

Some embodiments may include controlling, by the controller, the fluid pulse generator to puff the fluid pulse upon verification that the device is horizontal, for example, as described hereinabove.

Some embodiments may include preventing, by the controller, the fluid pulse generator from puffing the fluid pulse if the device is not horizontal, for example, as described hereinabove.

Some embodiments may include determining, by the processing unit, based on readings of one or more movement/accelerometer sensors, whether a subject's right eye or a subject's left eye is being tested by the device, for example, as described hereinabove.

Some embodiments may include determining, by the processing unit, based on readings of one or more movement/accelerometer sensors, whether or not the device is stationary, for example, as described hereinabove.

Some embodiments may include controlling, by the controller, the fluid pulse generator to puff the fluid pulse upon verification that the device is stationary, for example, as described hereinabove.

Some embodiments may include preventing, by the controller, the fluid pulse generator from puffing the fluid pulse if the device is not stationary, for example, as described hereinabove.

Some embodiments may include determining, by the processing unit, based on readings of one or more movement/accelerometer sensors, that the fluid pulse generator has puffed the fluid pulse, for example, as described hereinabove.

Some embodiments may include controlling, by the controller, light condition control means so as to cause the subject's eye to be exposed to substantially the same lighting conditions each time one or more image frames is being captured, for example, as described hereinabove.

Some embodiments of the present invention are described above with reference to flowchart illustrations and/or portion diagrams of methods, apparatus (systems) and computer program products according to embodiments of the invention. It will be understood that each portion of the flowchart illustrations and/or portion diagrams, and combinations of portions in the flowchart illustrations and/or portion diagrams, can be implemented by computer program instructions. These computer program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or portion diagram or portions thereof.

These computer program instructions can also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or portion diagram portion or portions thereof. The computer program instructions can also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or portion diagram portion or portions thereof.

The aforementioned flowchart and diagrams illustrate the architecture, functionality, and operation of possible implementations of systems, methods and computer program products according to various embodiments of the present invention. In this regard, each portion in the flowchart or portion diagrams can represent a module, segment, or portion of code, which includes one or more executable instructions for implementing the specified logical function(s). It should also be noted that, in some alternative implementations, the functions noted in the portion can occur out of the order noted in the figures. For example, two portions shown in succession can, in fact, be executed substantially concurrently, or the portions can sometimes be executed in the reverse order, depending upon the functionality involved. It will also be noted that each portion of the portion diagrams and/or flowchart illustration, and combinations of portions in the portion diagrams and/or flowchart illustration, can be implemented by special purpose hardware-based systems that perform the specified functions or acts, or combinations of special purpose hardware and computer instructions.

In the above description, an embodiment is an example or implementation of the invention. The various appearances of “one embodiment”, “an embodiment”, “certain embodiments” or “some embodiments” do not necessarily all refer to the same embodiments. Although various features of the invention can be described in the context of a single embodiment, the features can also be provided separately or in any suitable combination. Conversely, although the invention can be described herein in the context of separate embodiments for clarity, the invention can also be implemented in a single embodiment. Certain embodiments of the invention can include features from different embodiments disclosed above, and certain embodiments can incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A device for a non-contact measurement of an intraocular pressure (IOP) of an eye of a subject, the device comprising:
a frame;
a conduit connected to the frame and aligned along a conduit axis;
at least one camera connected to the frame and configured to capture one or more image frames, each comprising an image of a side view of a cornea of the subject's eye;
a fluid pulse generator in fluid communication with the conduit, wherein the fluid pulse generator is configured to generate a fluid pulse and to puff the fluid pulse through the conduit; and
a controller comprising a processing unit, wherein the processing unit is configured to:
receive the one or more image frames;
determine one or more fluid pulse pressure values; and
determine the IOP value of the subject's eye based on at least one of the one or more image frames and at least one of the one or more fluid pressure values.

2. (canceled)

3. (canceled)

4. The device of claim 1, wherein the camera is connected to the frame such that a field-of-view of the camera is located along an axis that is perpendicular to the conduit axis and is substantially parallel to a tangent to the cornea when the conduit is aligned with the cornea along the conduit axis.

5. The device of claim 1, wherein the processing unit is further configured to determine the one or more fluid pulse pressure values based on readings of a pressure sensor configured to measure a fluid pulse pressure within the conduit.

6. The device of claim 1, wherein the processing unit is further configured to determine the one or more fluid pulse pressure values based on a known pressure-time variation pattern of the fluid pulse and a time point at which the fluid pulse generator has puffed the fluid pulse through the conduit.

7. The device of claim 1, further comprising:
a first camera configured to capture one or more image frames of the side view of the cornea; and
a second camera configured to capture, at a predefined time shift with respect to the first camera, one or more image frames of the side view of the cornea; and
wherein the processing unit is configured to combine the one or more image frames captured by the first camera with the one or more image frames captured by the second camera into a single image dataset.

8. (canceled)

9. The device of claim 1, further comprising a conduit positioning mechanism configured to at least one of:
controllably move the conduit with respect to the frame along the conduit axis;
controllably move the conduit with respect to the frame in one or more directions that are perpendicular to the conduit axis; and
controllably rotate the conduit with respect to the frame about at least one axis that is perpendicular to the conduit axis.

10. The device of claim 1, wherein the one or more image frames further comprise an image of an outlet conduit end, and wherein the processing unit is further configured to determine a distance between the outlet conduit end and the cornea based on the one or more image frames.

11. The device of claim 10, wherein the controller is configured to:
control the fluid pulse generator to puff the fluid pulse if the outlet conduit end is at a predefined distance from the cornea; and
prevent the fluid pulse generator from puffing the fluid pulse if the outlet conduit end is not at a predefined distance from the cornea.

12. The device of claim 10, wherein the controller is further configured to control the conduit positioning mechanism so as to position the outlet conduit end at a predefined distance from the cornea.

13. The device of claim 1, comprising an additional camera connected to the frame and configured to capture one or more additional image frames, each comprising an image of a front view of the cornea of the subject's eye.

14. The device of claim 13, wherein the processing unit is configured to verify that the conduit is radial to a center of the cornea based on the one or more additional image frames and a known position of the conduit with respect to the frame.

15. The device of claim 14, wherein the controller is further configured to:
control the fluid pulse generator to puff the fluid pulse upon the verification that the conduit is radial to the center of the cornea; and
prevent the fluid pulse generator from puffing the fluid pulse if the conduit is not aligned with the center of the cornea.

16. (canceled)

17. (canceled)

18. (canceled)

19. (canceled)

20. The device of claim 1, further comprising one or more movement sensor sensors connected to the frame, wherein the processing unit is further configured, based on readings of the one or more movement sensors, to at least one of:
determine an orientation of the device in a reference coordinate system;
determine whether a subject's right eye or a subject's left eye is being tested by the device;
determine whether or not the device is stationary; and
determine that the fluid pulse generator has puffed the fluid pulse.

21. The device of claim 20, wherein the controller is further configured to:
control the fluid pulse generator to puff the fluid pulse upon verification that the device is horizontal; and
prevent the fluid pulse generator from puffing the fluid pulse if the device is not horizontal.

22. The device of claim 20, wherein the controller is further configured to:
control the fluid pulse generator to puff the fluid pulse upon verification that the device is stationary; and
prevent the fluid pulse generator from puffing the fluid pulse if the device is not stationary.

23. The device of claim 1, wherein the fluid pulse generator comprises:
a pump;
a first chamber downstream of the pump and in fluid communication with the pump;
a second chamber downstream of the first chamber and in fluid communication with the first chamber, wherein the second chamber comprises an outlet aperture;
a first valve downstream of the pump and upstream of the first chamber; and
a second valve downstream of the first chamber and upstream of the second chamber.

24. The fluid pulse generator of claim 23, wherein a volume of the first chamber, a volume of the second chamber, a cross section of the second valve, cross-sections of flow tubes, and a speed of opening the second valve are set to provide a desired pressure-time variation pattern a fluid pulse being puffed from the fluid pulse generator.

25. The fluid pulse generator of claim 23, further comprising:
a first pressure sensor configured to measure a fluid pressure in the first chamber; and
a second pressure sensor configured to measure a fluid pressure in the second chamber.

26. The fluid pulse generator of claim 25, wherein the pump, the first valve and the second valve are controllable by a controller based on readings of the first pressure sensor and the second pressure sensor.

27. The fluid pulse generator of claim 23, wherein the pump, the first valve and the second valve are controllable by a controller based on a predefined pump flowrate, a predefined speed of opening and closing of first valve, and a predefined speed of opening and closing of second valve.

28-52. (canceled)
